(19) United States
(12) Patent Application Publication
(10) Pub. No.: US 2023/0312872 A1
(43) Pub. Date: Oct. 5, 2023

(54) PLASTICIZERS FOR CELLULOSE ESTERS

(71) Applicant: 3M INNOVATIVE PROPERTIES COMPANY, St. Paul, MN (US)

(72) Inventors: Kevin M. Lewandowski, Inver Grove Heights, MN (US); Dean M. Moren, North St. Paul, MN (US)

(21) Appl. No.: 18/017,328

(22) PCT Filed: Jul. 6, 2021
(86) PCT No.: PCT/IB2021/056059

§ 371 Date: Jan. 20, 2023

Related U.S. Application Data
(60) Provisional application No. 63/058,164, filed on Jul. 29, 2020.

(51) Int. Cl. C08K 5/11; C07C 67/313

(52) U.S. Cl. CPC C08K 5/11; C07C 67/313

(57) ABSTRACT

A plasticizer composition that includes a) a cellulose ester polymer, and b) a plasticizer, where the plasticizer is compatible with the cellulose ester polymer. The plasticizer is a compound represented by the Formula (I). In Formula (I), R₁ is an alkyl group having from 1 to 4 carbons, inclusive, R₂ is a CH₃— group, a CH₃CH₂— group, or an R₁O— group, and each R₃ is independently an alkyl group having from 1 to 4 carbons, inclusive. Articles that includes the plasticizer composition and methods of preparing the plasticizer composition are provided.

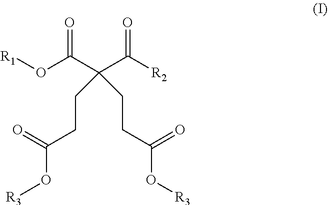

### TECHNICAL FIELD

Polymeric compositions that include acetoacetate ester compounds, methods of preparing the acetoacetate ester compounds, and articles that include the acetoacetate ester compounds are provided.

### BACKGROUND

Various ester compounds may be used as plasticizers in polymeric compositions. These plasticizers include, for example, alkyl phthalates (e.g., diethyl phthalate) and adipate esters. In recent years, various plasticizers based on renewable materials have been introduced. These plasticizers include those based on citric acid (e.g., from Vertellus), succinic acid (e.g., from BioAmber and Myriant), vegetable oil (e.g., from Danisco and Dow Chemical Co.), isosorbide (e.g., from Roquette), and levulinic acid (e.g., from Segetis).

### SUMMARY

Polymeric compositions are provided that include an acetoacetate ester plasticizer compound. The plasticizer has low odor, good compatibility with cellulose acetate, and can be formed from renewable resources. Further, the plasticizer can be used at temperatures often encountered during high-temperature processing of the polymeric compositions.

In a first aspect, a plasticizer composition is provided that includes a) a cellulose ester polymer, and b) a plasticizer, wherein the plasticizer is compatible with the cellulose ester polymer. The plasticizer is a compound represented by the Formula (I).

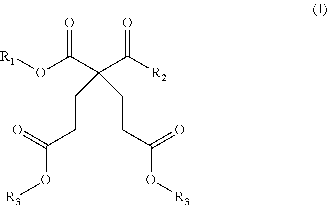

In Formula (I), R₁ is an alkyl group having from 1 to 4 carbons, inclusive, R₂ is a CH₃— group, a CH₃CH₂— group, or an R₁O— group, and each R₃ is independently an alkyl group having from 1 to 4 carbons, inclusive.

In a second aspect, an article is provided that includes a plasticizer composition. The plasticizer composition is the same as described above.

In a third aspect, a method of preparing a plasticizer composition is provided. The plasticizer composition is the same as described above.

Features and advantages of the present disclosure will be further understood upon consideration of the detailed description as well as the appended claims.

### DETAILED DESCRIPTION

There is interest among manufacturers in identifying phthalate-free compositions to accommodate possible regulatory changes and to increase the sustainability of various products including polymeric materials. The non-phthalate plasticizers of the present disclosure, i.e., acetoacetate ester compounds, have at least the following desirable characteristics: compatibility with cellulose acetate (up to at least 20 weight percent), effective reduction in glass temperature at the typical concentration range, a boiling point of at least 290° C., high retention under product-use conditions, low viscosity, high thermal stability (e.g., low formation of acidic species under processing conditions), low color, and low cost.

Polymeric compositions are provided herein that include a polymeric material plus an acetoacetate ester compound. The acetoacetate ester compound can function as a plasticizer for various compatible polymeric materials such as, for example, cellulose esters, e.g., cellulose acetates.

The terms “a”, “an”, “the”, “at least one”, and “one or more” are used interchangeably.

The term “and/or” means one or both such as the expression A and/or B refers to A alone, B alone, or both A and B.

The terms “polymeric material” and “polymer” are used interchangeably and can refer to a homopolymer, copolymer, terpolymer, and the like.

The term “cellulose triester polymer” as used herein refers to a cellulose acetate in which at least 92% of the hydroxyl groups are acetylated.

The term “secondary cellulose ester polymer” as used herein refers to a cellulose acetate in which about 76 percent of the hydroxyl groups are acetylated.

The term “alky” refers to a monovalent radical of an alkane. Suitable alkyl groups can have up to 4 carbon atoms, up to 3 carbon atoms, up to 2 carbon atoms, or up to 1 carbon atom.

The terms “weight percent” and “wt. %” are used interchangeably.

A polymeric composition, also referred to herein as a “plasticizer composition,” is provided that includes a) a plasticizer and b) a polymeric material, wherein the plasticizer is compatible with the polymeric material. The plasticizer is a compound of Formula (I).

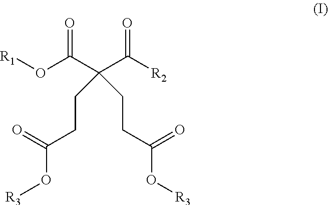

In Formula (I), R₁ is an alkyl group having from 1 to 4 carbons, inclusive, R₂ is a CH₃— group, a CH₃CH₂— group, or an R₁O— group, and each R₃ is independently an alkyl group having from 1 to 4 carbons, inclusive. The compound of Formula (I) is typically a liquid (e.g., oil) or low-melting solid at room temperature (e.g., about 20° C. to about 25° C.).

Provided herein are a class of multifunctional ester compounds of Formula (I) that may be derived from simple acetoacetate or malonate esters and alkyl acrylates (Scheme 1).

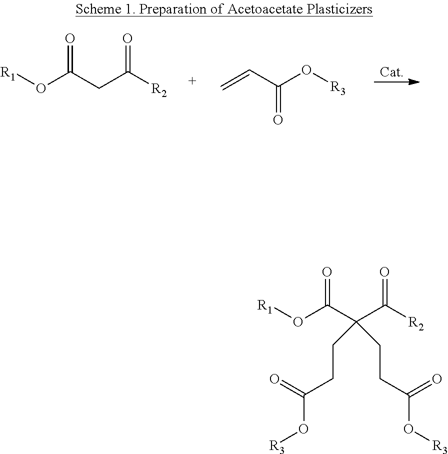

Referring to Scheme 1, the Michael acceptor (i.e., acetoacetate or malonate) is reacted with an alkyl acrylate (e.g., methyl acrylate) or a mixture of alkyl acrylates (e.g., methyl acrylate and ethyl acrylate, methyl acrylate and tert-butyl acrylate), in the presence of a basic catalyst. R₁ is a short-alkyl ester (i.e., alkyl group is methyl through butyl), R₂ is methyl, ethyl, or an O—R₁ group, and R₃ is an alkyl group, preferably methyl through butyl. In some embodiments, a single molecule of Formula (I) may include R₃ groups that are not the same, e.g., one R₃ group is methyl and one R₃ group is ethyl. In some embodiments, a single molecule of Formula (I) may include R₃ groups that are the same, e.g., R₃ groups are both methyl. Common acetoacetate ester building blocks include, for example, methyl, ethyl, and tert-butyl acetoacetate. Compounds where R₂ is ethyl include, for example, methyl 3-oxopentanoate and ethyl 3-oxopentanoate. Commercially available malonate derivatives include, for example, dimethyl malonate and diethyl malonate. Useful acrylates include methyl acrylate, ethyl acrylate, and butyl acrylate. If the ester groups in the compounds are too long (e.g., greater than butyl or if there is more than one butyl group in the plasticizer), the compatibility of the compounds with cellulose acetate is reduced. Therefore, the most favorable compounds are ones where R₁ and R₃ are methyl and/or ethyl groups.

The reaction to produce the plasticizers is well known and can be accelerated with a number of basic catalysts, including, for example, sodium metal, alkoxides, and tertiary amines. Tertiary amines that may be useful in some embodiments may include, for example, 1,8-diazabicyclo[5.4.0] undec-7-ene and 1,5-diazabicyclo[4.3.0]non-5-ene, reagents commercially available from Alfa Aesar, Ward Hill, MA, USA. The reaction results in high yields of products that can be isolated easily in high purity either with or without distillation. It has been observed that these resulting multi-functional esters, i.e., plasticizers, display a combination of useful properties as plasticizers for cellulose acetate including, for example, excellent compatibility, high boiling point (i.e., generally 370° C. to 450° C. at a pressure of 760 mm Hg) resulting in low odor and excellent high-temperature performance, and good plasticizer efficiency.

The plasticizer compound of Formula (I) can be combined with a compatible polymeric material to provide a plasticizer composition. As used herein, the term “compatible” refers to a polymeric material that is miscible with the compound of Formula (I). That is, the compound of Formula (I) is soluble in the polymeric material, thus a mixture of the polymeric material and the compound of Formula (I) forms a single phase. Compatibility can be determined, for example, by measuring the haze of a film prepared from a mixture of the polymeric material with the compound of Formula (I). One suitable method of measuring haze is described in the Example section (see Film Haze Measurement). A low haze value (e.g., less than 5, less than 4, less than 3, less than 2, or even less than 1) is typically associated with a mixture having compatible components.

In some preferred embodiments, the polymeric material is a cellulose ester (e.g., a reaction product of cellulose and a carboxylic acid). Cellulose esters useful in embodiments of the present disclosure may include cellulose triesters and secondary cellulose esters, such as, for example, cellulose triacetate, cellulose tripropionate, cellulose tributyrate, cellulose acetate, cellulose propionate, cellulose acetate propionate, cellulose butyrate, cellulose tributryrate, and cellulose acetate butyrate. The various cellulose esters can be prepared with differing solubility depending on the number of hydroxyl groups present using methods known to those of skill in the relevant arts. A variety of cellulose esters are commercially available from Eastman Chemical Company, Kingsport, TN, USA. In some preferred embodiments, the cellulose ester includes a cellulose acetate.

In some embodiments, the plasticizer composition may contain at least 5 wt. %, at least 10 wt. %, or at least 15 wt. % of the plasticizer. In some embodiments, the plasticizer composition may contain less than 95 wt. %, less than 90 wt. %, or less than 80 wt. % of the plasticizer. In some embodiments, the plasticizer composition may contain 5 wt. % to 95 wt. %, 10 wt. % to 90 wt. %, or 15 wt. % to 85 wt. % of the plasticizer.

In some embodiments, the plasticizer composition may contain at least 5 wt. %, at least 10 wt. %, or at least 15 wt. % of the polymer. In some embodiments, the plasticizer composition may contain less than 95 wt. %, less than 90 wt. %, or less than 80 wt. % of the polymer. In some embodiments, the plasticizer composition may contain 5 wt. % to 95 wt. %, 10 wt. % to 90 wt. %, or 15 wt. % to 85 wt. % of the polymer.

In some embodiments such as, for example, film applications, the amount of plasticizer is preferably more than 5 wt. %, optionally more than 7.5 wt. %, or optionally more than 10 wt. % of the total weight of the plasticizer composition. In these embodiments the amount of plasticizer is preferably less than 50 wt. %, optionally less than 30 wt. %, or optionally less than 25 wt. % of the total weight of the plasticizer composition. In these embodiments the amount of plasticizer is preferably 5 wt. % to 50 wt. %, optionally 7.5 wt. % to 30 wt. %, or optionally 10 wt. % to 25 wt. % (e.g., 13 wt. % to 20 wt. %) of the total weight of the plasticizer composition.

In some embodiments, the weight percent ratio of cellulose ester polymer to plasticizer in the plasticizer composition is 1:1 to 25:1, optionally 1:1 to 10:1 (e.g., 3.5:1 to 7:1), optionally 1:1 to 5:1, optionally 1:1 to 4:1, optionally 1:1 to 3:1, or optionally 1:1 to 2.5:1.

In some embodiments, other components may be added to the disclosed plasticizer compositions. Such optional components may include, but are not limited to, anti-blocking agents, anti-slip agents, fillers, nucleating agents, thermal stabilizers, light stabilizers, lubricants, pigments, colorants, anti-oxidants, anti-static agents, flame retardants, melt strength enhancers, impact modifiers, and the like. The use of any of these additional optional components may be desirable to provide compositions for specific applications.

In some embodiments, the plasticizers of Formula (I) may be used in combination with one or more other types of plasticizers such as, for example: aromatic carboxylate esters (e.g., phthalic acid di-C1-12 alkyl esters such as dimethyl phthalate, diethyl phthalate, dibutyl phthalate, dihexyl phthalate, dioctyl phthalate anddi-2-ethylhexyl phthalate; phthalic acid C1-6 alkoxy C1-12 alkyl esters such as dimethoxyethyl phthalate; phthalic acid C1-12 alkylaryl-C1-3 alkyl esters such as butyl benzyl phthalate; C1-6 alkylphthalyl C2-4 alkylene glycolates such as ethylphthalyl ethylene glycolate and butylphthalyl butylene glycolate; trimellitic acid tri-C1-12 alkyl esters such as trimethyl trimellitate, triethyl trimellitate, trioctyl trimellitate and tri-2-ethylhexyl trimellitate; pyromellitic acid tetra-C1-12 alkyl esters such as tetraoctyl pyromellitate); fatty acid esters (e.g., adipic acid esters such as dibutyl adipate, dioctyl adipate, butoxyethoxyethylbenzyl adipate and dibutoxyethoxyethyl adipate); azelaic acid esters such as diethyl azelate, dibutyl azelate and dioctyl azelate; sebacic acid esters such as dibutyl sebacate and dioctyl sebacate; butyl oleate; methylacetyl ricinoleate; lower fatty acid esters of polyhydric alcohols (e.g., glycerin, trimethylol propane, pentaerythritol, sorbitol; triacetin, diglycerine tetraacetate); glycol esters (e.g., dipropylene glycol dibenzoate); citric acid esters (e.g., acetyl tributyl citrate); amides (e.g., N-butyl benzene sulfonamide); and ester oligomers (e.g., caprolactone oligomer).

The plasticizer compositions can be prepared by a variety of methods. Examples of suitable mixing methods include admixing, melt blending, dry blending, and combinations thereof. Blending may be performed, for example, using a melt extruder, a kneader extruder, a roll mill, a high shear mixer, a twin-screw compounder, and combinations thereof. In some embodiments, the plasticizer compositions may also be prepared by dissolving the polymer and plasticizer in a suitable solvent (e.g., acetone tetrahydrofuran, methylene chloride) by methods known to those of skill in the art.

The plasticizer compositions can be formed into a wide variety of forms including, for example, shaped articles, such as films, molded objects, and fibers.

Objects and advantages of this disclosure are further illustrated by the following non-limiting Examples section, but the particular materials and amounts thereof recited in these Examples, as well as other conditions and details, should not be construed to unduly limit this disclosure.

### EXAMPLES

Unless otherwise noted or readily apparent from the context, all parts, percentages, ratios, etc. in the Examples and the rest of the specification are by weight.

TABLE 1
Materials Used in the Examples
Description and Source

Methyl acetoacetate, obtained from Oakwood Products, Inc., Estill, SC,
USA
Ethyl acetoacetate, obtained from Alfa Aesar, Ward Hill, MA, USA
tert-Butyl acetoacetate, obtained from Alfa Aesar, Ward Hill, MA, USA
Methyl acrylate, obtained from Alfa Aesar, Ward Hill, MA, USA
Ethyl acrylate, obtained from Alfa Aesar, Ward Hill, MA, USA
Butyl acrylate, obtained from Alfa Aesar, Ward Hill, MA, USA
Dimethyl malonate, obtained from Oakwood Products, Inc., Estill, SC,
USA
1,8-diazabicyclo[5.4.0] undec-7-ene (“DBU”), obtained from Alfa Aesar,
Ward Hill, MA, USA
Cellulose acetate, abbreviated “CA”, obtained as cellulose acetate 398-30
from Eastman Chemical Company, Kingsport, TN, USA
Diethyl phthalate (“DEP”), obtained from Millipore Sigma, Burlington,
MA, USA
IRGANOX 1076, obtained from BASF, Freeport, TX, USA
ULTRANOX 626, obtained from Addivant, Danbury, CT, USA

### Test Methods:

### Test Method 1: Differential Scanning Calorimeter (“DSC”) Analysis of Films

The specimens were prepared for thermal analysis by weighing and loading the material into TA Instruments aluminum DSC sample pans (Thermal Analysis T080715). The specimens were analyzed using a Modulated Differential Scanning Calorimeter (MDSC) (TA Instruments Q2500, New Castle, DE) utilizing a heat-cool-heat profile in temperature modulated mode (−60 to 250° C. at 5° C./min with a modulation amplitude of 0.796° C. and a period of 60 sec). The midpoint (half height) was recorded for the second heat profile.

### Test Method 2: Film Haze Measurement

The film haze was measured using an Ultrascan Pro Spectrophotometer (Hunter Associates Laboratory, Reston, Va.). The instrument was standardized with a diffuse/8° white tile instrument standard (Hunter Associates Laboratory). The film sample was then placed flush with the transmission port on the inside of the instrument. A measurement was taken with a white tile standard followed by a light trap (Hunter Associates Laboratory) on the outside of the unit, and the percent haze was recorded.

### Test Method 3: Thermogravimetric Analysis (“TGA”) of Plasticizers

The weight loss of the plasticizers was measured by TGA. Approximately 17-25 milligrams of a sample was placed in a standard aluminum pan and heated to 300° C. at a rate of 10° C./min using a Model TGA 5500 (T.A. Instruments, New Castle, DE, USA). The weight loss of each sample was determined at 200° C. and 250° C.

### Test Method 4: Measurement of Film Physical Properties

The tensile strength, percent elongation, and modulus of the films were measured using an Instron 3365 tensile testing machine with a 500 N load cell (Instron, Norwood, MA., USA). Specimens for this test were 2.54 centimeters wide and 15 centimeters long. An initial jaw separation of 5.08 centimeters and a crosshead speed of 12.7 centimeters per minute were used.

### Test Method 5: Film Weight Loss

Polymer film (0.4 g; several layers of 0.07 mm thick film) was placed in an aluminum pan. The sample was placed in an oven at 120° C. The weight was recorded after removal from the oven at 3, 24, and 96 hours.

### Preparatory Examples (“PE”)

### PE 1: 4-acetyl-4-(methoxycarbonyl)-heptanedioic acid 1,7-dimethyl ester

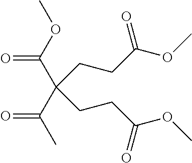 [SMILES: COC(=O)CCC(CCC(=O)OC)(C(C)=O)C(=O)OC]

A mixture of methyl acetoacetate (Oakwood Products, Inc., Estill, SC, 20.00 g, 172 mmol), methyl acrylate (Alfa Aesar, Ward Hill, MA 33.36 g, 388 mmol), and 1,8-diazabicyclo[5.4.0] undec-7-ene (DBU, Alfa Aesar, 0.25 g, 2 mmol) was heated at 60° C. for six hours. The mixture was diluted with ethyl acetate (200 mL) and washed with 1.0 M aqueous hydrochloric acid. The organic layer was then dried over magnesium sulfate. The solvent and excess methyl acrylate were then removed under vacuum. The residual oil was then distilled under high vacuum. A colorless oil was obtained at 145-150° C. at 0.30 mmHg. After standing the oil crystallized into a solid (melting point 43-45° C.) with a yield of 41.56 g.

### PE 2: 4-acetyl-4-(methoxycarbonyl)-heptanedioic acid 1,7-dibutyl ester

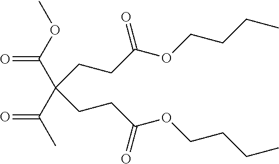 [SMILES: CCCCOC(=O)CCC(CCC(=O)OCCCC)(C(C)=O)C(=O)OC]

A mixture of methyl acetoacetate (20.00 g, 172 mmol), butyl acrylate (Alfa Aesar, 48.70 g, 380 mmol), and DBU (0.25 g, 2 mmol) was heated at 60° C. for six hours. The mixture was diluted with ethyl acetate (200 mL) and washed with 1.0 M aqueous hydrochloric acid. The organic layer was then dried over magnesium sulfate. The solvent was then removed under vacuum. The residual oil was then distilled under high vacuum. A colorless oil was obtained at 155-160° C. at 0.20 mmHg with a yield of 20.33 g.

### PE 3: 4-acetyl-4-(ethoxycarbonyl)-heptanedioic acid 1,7-dimethyl ester

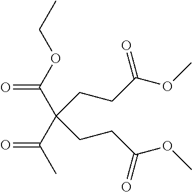 [SMILES: CCOC(=O)C(CCC(=O)OC)(CCC(=O)OC)C(C)=O]

A mixture of ethyl acetoacetate (Alfa Aesar, 40.00 g, 307 mmol), methyl acrylate (79.38 g, 922 mmol), and DBU (0.46 g, 3 mmol) was heated at 60° C. for three hours. The mixture was diluted with ethyl acetate (200 mL) and washed with 1.0 M aqueous hydrochloric acid. The organic layer was then dried over magnesium sulfate. The solvent and excess methyl acrylate were then removed under vacuum. The residual oil was then distilled under high vacuum. A colorless oil was obtained at 134-142° C. at 0.20 mmHg with a yield of 76.35 g.

### PE 4: 4-acetyl-4-(ethoxycarbonyl)-heptanedioic acid 1,7-diethyl ester

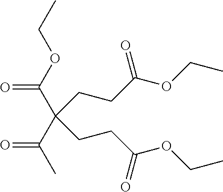 [SMILES: CCOC(=O)CCC(CCC(=O)OCC)(C(C)=O)C(=O)OCC]

A mixture of ethyl acetoacetate (40.00 g, 307 mmol), ethyl acrylate (Sigma Aldrich, Milwaukee, WI, 77.57 g, 775 mmol), and sodium metal (Alfa Aesar, 0.21 g, 9 mmol) was heated at 60° C. for four hours. Concentrated hydrochloric acid (1 mL) was added and the mixture was dried over magnesium sulfate. The mixture was then distilled under high vacuum. A colorless oil was obtained at 134-150° C. at 0.25 mmHg with a yield of 74.49 g.

### PE 5: 4-acetyl-4-(ethoxycarbonyl)-heptanedioic acid 1,7-dibutyl ester

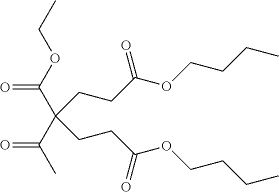 [SMILES: CCCCOC(=O)CCC(CCC(=O)OCCCC)(C(C)=O)C(=O)OCC]

A mixture of ethyl acetoacetate (15.00 g, 115 mmol), butyl acrylate (44.32 g, 346 mmol), and DBU (0.25 g, 2 mmol) was heated at 60° C. for four hours. The mixture was diluted with ethyl acetate (200 mL) and washed with 1.0 M aqueous hydrochloric acid. The organic layer was then dried over magnesium sulfate. The solvent was then removed under vacuum. The residual oil was then placed under high vacuum (100° C. at 0.20 mmHg) to remove residual butyl acrylate. A colorless oil was obtained with a yield of 39.46 g.

### PE 6: 4-acetyl-4-(tert-butoxycarbonyl)-heptanedioic acid 1,7-dimethyl ester

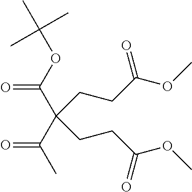 [SMILES: COC(=O)CCC(CCC(=O)OC)(C(C)=O)C(=O)OC(C)(C)C]

A mixture of tert-butyl acetoacetate (Alfa Aesar, 20.00 g, 126 mmol), methyl acrylate (22.78 g, 265 mmol), and DBU (0.20 g, 1 mmol) was heated at 60° C. for thirty hours. The mixture was diluted with ethyl acetate (200 mL) and washed with 1.0 M aqueous hydrochloric acid. The organic layer was then dried over magnesium sulfate. The solvent and excess methyl acrylate were then removed under vacuum. The residual oil was then placed under high vacuum (100° C. at 0.20 mmHg) to remove volatile by-products. A yellow oil was obtained with a yield of 37.40 g.

### PE 7: 4,4′-(methoxycarbonyl)-heptanedioic acid 1,7-dimethyl ester

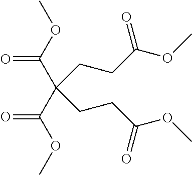 [SMILES: COC(=O)CCC(CCC(=O)OC)(C(=O)OC)C(=O)OC]

A mixture of dimethyl malonate (Oakwood Products, Inc., 15.00 g, 114 mmol), methyl acrylate (29.32 g, 340 mmol), and DBU (0.15 g, 1 mmol) was heated at 60° C. for four hours. The mixture was diluted with ethyl acetate (200 mL) and washed with 1.0 M aqueous hydrochloric acid. The organic layer was then dried over magnesium sulfate. The solvent and excess methyl acrylate were then removed under vacuum. The residual oil was then distilled under high vacuum. A colorless oil was obtained at 155-160° C. at 0.20 mmHg. The oil slowly crystallized into a waxy solid (melting point 54-56° C.) with a yield of 16.25 g.

### PE 8: 4.4′-(methoxycarbonyl)-heptanedioic acid 1,7-dibutyl ester

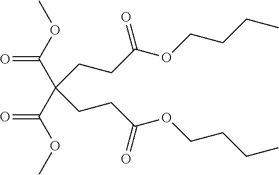 [SMILES: CCCCOC(=O)CCC(CCC(=O)OCCCC)(C(=O)OC)C(=O)OC]

A mixture of dimethyl malonate (10.00 g, 75 mmol), butyl acrylate (29.10 g, 227 mmol), and DBU (0.12 g, 1 mmol) was heated at 60° C. for four hours. The mixture was diluted with ethyl acetate (200 mL) and washed with 1.0 M aqueous hydrochloric acid. The organic layer was then dried over magnesium sulfate. The solvent was then removed under vacuum. The residual oil was then placed under high vacuum (150° C. at 0.20 mmHg) to remove volatile by-products. A yellow oil was obtained with a yield of 9.49 g.

### Thermogravimetric Analysis of Preparatory Examples

TABLE 2
Thermogravimetric Analysis of Plasticizers

| | Weight loss at 200° C.* | Weight Loss at 250° C.* |
|---|---|---|
| Plasticizer | (wt. %) | (wt. %) |
| DEP | 87.8 | 100 |
| PE 1 | 15.3 | 93.8 |
| PE 2 | 7.0 | 50.7 |
| PE 3 | 17.2 | 100 |
| PE 4 | 16.7 | 89.8 |
| PE 5 | 8.7 | 41.0 |
| PE 6 | 25.0 | 100 |
| PE 7 | 23.5 | 100 |
| PE 8 | 5.1 | 34.6 |

*Measured according to Test Method 3

The preparatory examples of this disclosure have reduced volatility at 200° C. compared to the commonly used DEP.

### Examples of Solvent Cast Cellulose Acetate Films

Cellulose acetate (“CA”), plasticizer, and acetone were placed in a glass jar (weights according to Table 3). The jar was capped and rolled at room temperature for 17 hours. A film was prepared from each solution by knife coating onto a polyester backing using a gap of 0.635 mm. After standing for 4 hours, the film (approximately 0.04-0.07 mm thick) was peeled from the backing. Comparative examples were performed using no plasticizer and diethyl phthalate (“DEP”). The glass transition temperature, haze, and physical properties of the films were determined according to test methods 1, 2, and 4, respectively.

TABLE 3
Solvent Cast Plasticized Cellulose Acetate Films

| | | | | | Plasti- | | |
|---|---|---|---|---|---|---|---|
| | | | Plasti- | | cizer | | |
| | CA | Plasti- | cizer | Acetone | in CA | Haze | Tg |
| Example | (grams) | cizer | (grams) | (grams) | (wt. %) | (%) | (° C.) |
| CE 1 | 10.00 | none | 0 | 50 | 0 | 1.9 | 196 |
| CE 2 | 8.20 | DEP | 1.80 | 50 | 18 | 1.3 | 128 |
| EX 1 | 8.20 | PE 1 | 1.80 | 50 | 18 | 1.1 | 119 |
| EX 2 | 8.20 | PE 2 | 1.80 | 50 | 18 | 1.2 | 144 |
| EX 3 | 4.50 | PE 3 | 0.50 | 25 | 10 | 1.0 | 148 |
| EX 4 | 4.25 | PE 3 | 0.19 | 25 | 15 | 0.9 | 136 |
| EX 5 | 8.20 | PE 3 | 1.80 | 50 | 18 | 1.1 | 125 |
| EX 6 | 4.00 | PE 3 | 1.00 | 25 | 20 | 1.0 | 117 |
| EX 7 | 3.75 | PE 3 | 1.25 | 25 | 25 | 1.2 | 102 |
| EX 8 | 3.50 | PE 3 | 1.50 | 25 | 30 | 1.1 | 92 |
| EX 9 | 8.20 | PE 4 | 1.80 | 50 | 18 | 1.1 | 126 |
| EX 10 | 8.20 | PE 5 | 1.80 | 50 | 18 | 1.2 | 149 |
| EX 11 | 8.20 | PE 6 | 1.80 | 50 | 18 | 0.7 | 156 |
| EX 12 | 8.20 | PE 7 | 1.80 | 50 | 18 | 1.4 | 128 |
| EX 13 | 8.20 | PE 8 | 1.80 | 50 | 18 | 1.3 | 132 |

TABLE 4
Film Properties of Solvent Cast Cellulose Acetate Films

| | Tensile Stress at | Elongation | Modulus |
|---|---|---|---|
| Example | Break (MPa) | (%) | (MPa) |
| CE 2 | 26.7 | 13.9 | 1300 |
| EX 1 | 18.7 | 13.8 | 790 |
| EX 2 | 22.7 | 22.2 | 873 |
| EX 5 | 27.6 | 17.5 | 1130 |
| EX 9 | 26.6 | 14.8 | 1200 |
| EX 10 | 23.1 | 17.1 | 1060 |
| EX 12 | 20.4 | 24.7 | 511 |
| EX 13 | 19.6 | 28.8 | 385 |

TABLE 5
Weight Loss of Solvent Cast Cellulose
Acetate Films After Heat Aging

| | Weight Loss at 120° C.* | | |
|---|---|---|---|
| | (wt %) | | |
| Example | 3 hr | 24 hr | 96 hr |
| CE 2 | 6.4 | 9.1 | 10.4 |
| EX 3 | 5.3 | 7.8 | 8.5 |
| EX 9 | 2.2 | 4.0 | 5.0 |
| EX 10 | 2.1 | 4.9 | 6.2 |
| EX 11 | 1.5 | 3.5 | 4.2 |
| EX 12 | 1.7 | 3.3 | 4.1 |
| EX 13 | 2.4 | 4.6 | 5.7 |

*Measured according to Test Method 5

### Examples of Thermal Compounding Cellulose Acetate and Melt Pressing to Form Films

Mixtures of cellulose acetate (13.94 g), plasticizer (3.06 g), Irganox 1076 (0.051 g) and Ultranox 626 (0.026 g) were placed in a Max 40 container (Flack Tek Inc., Landrum, SC) and speed mixed at 3500 rpm for one minute (DAC 150.1 FVZ-K Speed mixer, Flack Tek Inc.). The compounded mixtures were mixed in an Xplore Microcompounder MC15 (Xplore Instruments, The Netherlands) at a set temperature of 240° C. with a mixing speed of 100 rpm. After fully charging the compounded resin (17 g), the mixture was blended for three minutes, then drained from the melt mixer. Films were pressed between polyimide films by using 3.5 g of the polymer resin in a hot press (Carver 2699, Carver Inc., USA) with a clamp force of 6000 lbs at 230° C. to give a final film thickness of 0.1-0.2 mm. Haze and glass transition temperature of the films were determined according to Test Methods 1 and 2, respectively.

TABLE 6
Properties of Melt pressed Plasticized Cellulose Acetate Films

| | | Plasticizer | Haze | Tg |
|---|---|---|---|---|
| Example | Plasticizer | (wt %) | (%) | (° C.) |
| EX 14 | PE 1 | 18 | 1.0 | 122 |
| EX 15 | PE 2 | 18 | 99.0 | 147 |
| EX 16 | PE 3 | 18 | 1.6 | 134 |
| EX 17 | PE 4 | 18 | 1.0 | 133 |
| EX 18 | PE 5 | 18 | 38.5 | 152 |
| EX 19 | PE 6 | 18 | 1.7 | 144 |
| EX 20 | PE 7 | 18 | 0.9 | 126 |
| EX 21 | PE 8 | 18 | 51.1 | 147 |

The plasticizers having two butyl groups (e.g., from butyl acrylate), PE 2, PE 5, and PE 8, were cloudy and not fully compatible with cellulose acetate when prepared by thermal compounding.

All cited references, patents, and patent applications in the above application for letters patent are herein incorporated by reference in their entirety in a consistent manner. In the event of inconsistencies or contradictions between portions of the incorporated references and this application, the information in the preceding description shall control. The preceding description, given in order to enable one of ordinary skill in the art to practice the claimed disclosure, is not to be construed as limiting the scope of the disclosure, which is defined by the claims and all equivalents thereto.

1. A plasticizer composition comprising:
a cellulose ester polymer; and
a plasticizer comprising:
a compound represented by the formula 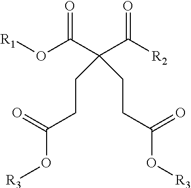
wherein
R₁ is an alkyl group having from 1 to 4 carbons, inclusive,
R₂ is a CH₃— group, a CH₃CH₂— group, or an R₁O— group, and
each R₃ is independently an alkyl group having from 1 to 4 carbons, inclusive.

2. The plasticizer composition of claim 1, wherein R₁ is a CH₃— group or a CH₃CH₂— group and each R₃ is independently a CH₃— group or a CH₃CH₂— group.

3. The plasticizer composition of claim 1, wherein the plasticizer has a boiling point of 370° C. to 450° C. at a pressure of 760 mm Hg.

4. The plasticizer composition of claim 1, wherein the cellulose ester polymer is selected from the group consisting of a cellulose triester polymer, a secondary cellulose ester polymer, and combinations thereof.

5. The plasticizer composition of claim 4, wherein the cellulose ester polymer comprises a cellulose acetate.

6. The plasticizer composition of claim 1, wherein the plasticizer composition comprises 5 wt. % to 95 wt. % of the plasticizer.

7. The plasticizer composition of claim 1, wherein the plasticizer composition comprises 5 wt. % to 50 wt. % of the plasticizer.

8. The plasticizer composition of claim 1, wherein the wt. % ratio of cellulose ester polymer to plasticizer in the plasticizer composition is 1:1 to 25:1.

9. An article comprising the plasticizer composition of claim 1.

10. The article of claim 9, wherein the article is selected from the group consisting of a film, a molded object, and a fiber.

11. A plasticizer composition comprising:
a cellulose ester polymer; and
a plasticizer comprising a reaction product of a compound represented by the formula 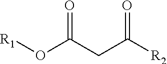
wherein
R₁ is an alkyl group having from 1 to 4 carbons, inclusive, and
R₂ is a CH₃— group, a CH₃CH₂— group, or an R₁O— group, and
a compound represented by the formula 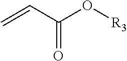
wherein
R₃ is an alkyl group having from 1 to 4 carbons, inclusive.

12. The plasticizer composition of claim 11, wherein the cellulose ester polymer is selected from the group consisting of a cellulose triester polymer, a secondary cellulose ester polymer, and combinations thereof.

13. The plasticizer composition of claim 12, wherein the cellulose ester polymer comprises a cellulose acetate.

14. The plasticizer composition of claim 11, wherein the composition further comprises a compound represented by the formula 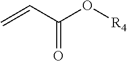
wherein
R₄ is an alkyl group having from 1 to 4 carbons, inclusive, different than R₃.

15. A method of making a plasticizer composition, the method comprising:
combining of a compound represented by the formula 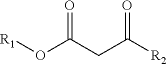
wherein
R₁ is an alkyl group having from 1 to 4 carbons, inclusive, and
R₂ is a CH₃— group, a CH₃CH₂— group, or an R₁O— group, and
a compound represented by the formula 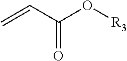
wherein
R₃ is an alkyl group having from 1 to 4 carbons, inclusive to form a mixture; and
adding a cellulose ester polymer to the mixture to form the plasticizer composition.

16. The method of claim 15, further comprising adding a catalyst to the mixture.

17. The method of claim 16, wherein the catalyst comprises a base catalyst.

18. The method of claim 17, wherein the base catalyst is selected from the group consisting of sodium metal, an alkoxide, a tertiary amine, and combinations thereof.

19. The method of claim 15, wherein the plasticizer composition comprises 5 wt. % to 95 wt. % of the mixture.

20. The method of claim 15, wherein the plasticizer composition comprises 5 wt. % to 50 wt. % of the mixture.

21. The method of claim 15, wherein the wt. % ratio of cellulose ester polymer to plasticizer in the plasticizer composition is 1:1 to 25:1.
